(19) United States
(12) Patent Application Publication
(10) Pub. No.: US 2017/0290771 A1
(43) Pub. Date: Oct. 12, 2017

(54) BIODEGRADABLE IN SITU FORMING MICROPARTICLES AND METHODS FOR PRODUCING THE SAME

(71) Applicant: Southern Research Institute, Birmingham, AL (US)

(72) Inventor: Michael E. Hudson, Hayden, AL (US)

(21) Appl. No.: 15/096,917

(22) Filed: Apr. 12, 2016

(51) Int. Cl. A61K 9/107; A61K 9/16

(52) U.S. Cl. CPC A61K 9/1075; A61K 9/1682

(57) ABSTRACT

In accordance with the present invention, disclosed herein is a kit comprising a first mixture comprising a pharmaceutically acceptable polar liquid and a fully or nearly fully dissolved biodegradable polymer; a second mixture comprising a pharmaceutically acceptable non-polar liquid; and a mixing device configured to mix the first mixture and second mixture, thereby forming a droplet comprising the biodegradable polymer. Also disclosed herein are methods of producing a microparticle in vivo in a subject.

### BACKGROUND

A variety of approaches have been developed for administering a biologically active agent to a patient in a continuous or sustained manner. However, currently available approaches suffer from one or more disadvantages or limitations.

In many conventional controlled release systems, the active agents are incorporated into solid, monolithic polymeric matrices. Often, however, the matrices need to be surgically implanted into patients' bodies to achieve a controlled release of active agents. As a result, in some instances, the matrices' shapes and sizes as well as the need for the surgical implantation can lead to patient discomfort and complications.

Other conventional controlled release systems are based on preformed microsphers, for example, RISPERDAL® CONSTA® (risperidone), a long-acting injectable medication that comprises a combination of extended-release microspheres for injection and a diluent for parenteral use. However, such preformed microsphers require complex manufacturing processes with multiple unit operations that affect an economical value of the drug. Additional drawbacks rise from a difficulty to manufacture the drug aseptically. Moreover, the needle can be easily blocked during injection due to solids being suspended in an injection vehicle.

In recent years, in situ forming implants, such as gel formulations, for example ELIGARD® have been developed. However, when injected, the in situ formed implants may have an irregular or uncontrolled shape that makes it hard to control the release of a biologically active agent from the implant.

An improved method for sustained release of a biologically active agent would be desirable.

Accordingly, such compositions, methods for making the same, and kits are described herein.

### SUMMARY OF THE INVENTION

Disclosed herein is a kit comprising: a) a first mixture comprising a pharmaceutically acceptable polar liquid and a fully or nearly fully dissolved biodegradable polymer; b) a second mixture comprising a pharmaceutically acceptable non-polar liquid; and c) a mixing device configured to mix the first mixture and second mixture, thereby forming a droplet comprising the biodegradable polymer.

Also disclosed herein is a kit comprising: a) a biologically active agent; b) a pharmaceutically acceptable carrier capable of delivering the biologically active agent; c) a first mixture comprising a pharmaceutically acceptable polar liquid and a fully or nearly fully dissolved biodegradable polymer; d) a second mixture comprising a pharmaceutically acceptable non-polar liquid; e) and one or more mixing devices configured to mix the biologically active agent and the pharmaceutically acceptable carrier with the first mixture to form a primary suspension and then to mix the primary suspension with the second mixture, thereby forming at least one droplet comprising the biologically active agent.

Also disclosed herein is a method of producing a microparticle in vivo in a subject comprising the steps of: a) mixing a first mixture comprising a pharmaceutically acceptable polar liquid and a fully or nearly fully dissolved biodegradable polymer in the a pharmaceutically acceptable polar liquid and a second mixture comprising a pharmaceutically acceptable non-polar liquid in a mixing device, thereby forming a droplet comprising the biodegradable polymer; and b) prior to forming the microparticle, injecting the droplet into a subject with an injection device, thereby forming the microparticle in vivo in the subject.

Further disclosed herein is a method of producing a microparticle in vivo in a subject comprising the steps of: a) mixing a biologically active agent with a first solution comprising a pharmaceutically acceptable carrier to form a first composition; b) mixing the first composition with a second solution in a first mixing device, wherein the second solution comprises a biodegradable polymer and a pharmaceutically acceptable polar liquid to form a primary emulsion; c) mixing the primary emulsion with a second mixture comprising a pharmaceutically acceptable non-polar liquid in a second mixing device to form a droplet within a secondary emulsion; d) prior to forming the microparticle, injecting the droplet within the secondary emulsion into a subject with an injection device, thereby forming the microparticle in vivo in the subject.

Additional advantages will be set forth in part in the description which follows, and in part will be obvious from the description, or can be learned by practice of the aspects described below. The advantages described below will be realized and attained by means of the chemical compositions, methods, and combinations thereof particularly pointed out in the appended claims. It is to be understood that both the foregoing general description and the following detailed description are exemplary and explanatory only and are not restrictive of the invention, as claimed.

### DESCRIPTION OF THE FIGURES

The accompanying figures, which are incorporated in and constitute a part of this specification, illustrate several aspects, and together with the description, serve to explain the principles of the invention.

FIG. 1 shows an unmodified luer connector as a part of an exemplary mixing device.

FIG. 2 shows a reduced inner diameter (I.D) luer connector as a part of an exemplary mixing device.

FIG. 3 shows a central plate with holed luer connector as a part of an exemplary mixing device.

FIG. 4 shows a static mixer luer connector as a part of an exemplary mixing device.

FIG. 5 shows a schematic representation of a first exemplary process to form a composition capable of forming microparticles.

FIG. 6 shows a schematic representation of a second exemplary process to form a composition capable of forming microparticles

### DETAILED DESCRIPTION

The present invention can be understood more readily by reference to the following detailed description of the invention.

Disclosed herein are materials, kits, compounds, compositions, and components that can be used for, can be used in conjunction with, can be used in preparation for, or are products of the disclosed method and compositions. It is to be understood that when combinations, subsets, interactions, groups, etc. of these materials are disclosed that while specific reference of each various individual and collective combinations and permutation of these compounds cannot be explicitly disclosed, each is specifically contemplated and described herein. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

All publications mentioned herein are incorporated herein by reference to disclose and describe the methods and/or materials in connection with which the publications are cited.

### 1. DEFINITIONS

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical acceptable solvent” includes mixtures of two or more such solvents, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

By “sufficient amount,” “effective amount,” “sufficient time,” and “effective time,” means an amount and time needed to achieve the desired result or results, e.g., dissolve a portion of the polymer.

The term “flowable substance” refers to a substance having a viscosity what will permit displacement of the flowable material with or without application of pressure. A flowable delivery composition or a liquid delivery composition is manipulatable, is displaceable through a small to moderate sized orifice and may be shaped and molded with the tissue defect. Flowable compositions in this context include those having a consistence from that of an emulsion or suspension with a low viscosity or water-like consistency, to that of a high viscosity substance.

The term “biocompatible” refers a substance that is substantially non-toxic to a subject, such as, for example, a human. It is also understood that the term “biocompatible” as used herein means the biological response to the material or device is appropriate for the device's intended application in vivo. Any metabolites of these materials should also be biocompatible.

The term “mixture,” for example first mixture and second mixture, refers to a composition that can comprise one or more substances or materials. For example the term mixture is intended to include a composition with only one material, such as, for example, a composition of a non-polar liquid. The term mixture is also intended to include a composition with more than one material, such as, for example, a composition of a polar liquid and a biodegradable polymer. The term mixture includes homogeneous and heterogeneous mixtures.

“Biodegradable” generally refers to a biocompatible material that will degrade or erode under physiologic conditions to smaller units or chemical species that are, themselves, biocompatible or non-toxic to the subject and capable of being metabolized, eliminated, or excreted by the subject.

“Polymer” as used herein refers to any type of polymer including, for example, a homopolymer, a copolymer, a block copolymer, a random copolymer, and the like.

“Absorbable” as used herein means the complete degradation of a material in vivo and elimination of its metabolites from an animal or human subject.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

The terms “bioactive agent” or “biologically active agent” or active pharmaceutical ingredient can be used interchangeably and refer to an agent that has biological activity. The bioactive agent can include a compound of interest contained in or on the microparticle such as therapeutic or biologically active compounds that may be used internally or externally as a medicine for the treatment, diagnosis, cure, or prevention of a disease or disorder. The biological agent can be used to treat, diagnose, cure, mitigate, prevent (i.e., prophylactically), ameliorate, modulate, or have an otherwise favorable effect on a disease, disorder, infection, and the like. Examples can include, but are not limited to, drugs, small-molecule drugs, peptides, proteins, oligonucleotides. “Bioactive agent” includes a single such agent and is also intended to include a plurality of bioactive agents including, for example, combinations of 2 or more bioactive agents.

A “releasable bioactive agent” or a “releasable biological agent” is one that can be released from a disclosed microparticle. Bioactive agents also include those substances which affect the structure or function of a subject, or a pro-drug, which becomes bioactive or more bioactive after it has been placed in a predetermined physiological environment.

The term “microparticle” is used herein to include nanoparticles, microspheres, nanospheres, microcapsules, nanocapsules, and particles, in general. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. The term “microparticle” refers generally to particles that have sizes in the range of about 10 nanometers (nm) to about 2 mm (millimeters).

Unless stated to the contrary, species described herein comprise all possible individual isomers, e.g., each enantiomer and diastereomer, and a mixture of isomers, such as a racemic or scalemic mixtures. Enantiomeric species may exist in different isomeric or enantiomeric forms. Unless otherwise specified, enantiomeric species discussed herein without reference to their isomeric form shall include all various isomeric forms as well as racemic mixtures of isomeric forms. For example, reference to lactic acid shall herein include L-lactic acid, D-lactic acid, and racemic mixtures of the L- and D-isomers of lactic acid; reference to lactide shall herein include L-lactide, D-lactide, and DL-lactide (where DL-lactide refers to racemic mixtures of the L- and D-isomers of lactide); similarly, reference to poly(lactide) shall herein include poly(L-lactide), poly(D-lactide) and poly(DL-lactide); similarly, reference to poly(lactide-co-glycolide) will herein include poly(L-lactide-co-glycolide), poly(D-lactide-co-glycolide), and poly(DL-lactide-co-glycolide); and so on.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### 2. KIT

Disclosed herein is a kit that can provide the means for the production of a droplet and in turn a microparticle for controlled release delivery of a biologically active agent. It is desirable to have the ability to control the release kinetics of biologically active agent in vivo. One avenue to achieve such control is to control the microparticle sizes where the biologically active agent is released from. The kit disclosed herein has the capability to be modified for specific applications to produce a desired size microparticle with a desired release kinetic. The kit disclosed herein achieves this by being configured to produce a droplet comprising a biodegradable polymer and the biologically active agent outside the body. The kit is configured to control the size and size distribution of the droplet. The kit can then be modified to inject the droplet into the body of a subject. Microparticles are then formed in vivo. The size of the microparticle corresponds to the size of the droplet. That is the larger the droplet the larger the microparticle.

The kit is configured to combine a first mixture comprising a pharmaceutically acceptable polar liquid and a fully or nearly fully dissolved biodegradable polymer, and a second mixture comprising a pharmaceutically acceptable non-polar liquid. A mixing device combines the first and second mixture to form a droplet, wherein the mixing device can be configured to produce a droplet of a predetermined size. An injection device, which can be a mixing device that has been modified with a needle that can be a part of the kit is then used to inject the droplet into the body of a subject. A microparticle is formed in vivo, which corresponds in size to the droplet. The size of the microparticle can control the release of a biologically active agent that is present in the microparticle. The microparticle is formed when the droplet comes into contact with an aqueous medium such as a body fluid, the polar solvent dissipated or diffuses into the aqueous medium. In certain aspects, as the solvent dissipates into the surrounding body fluids, the polymer precipitates to form a microparticle, and the active agent can become trapped or encapsulated throughout the microparticle (polymer matrix). In certain aspects, the release of the active agent from the microparticle polymeric matrix depends on the type of drug or medicament. In some aspects, of the current description, the release of the biologically active agent can be manipulated by changing a concentration and/or type of a biodegradable polymer present in the emulsion. In yet other aspects, the release of the biologically active agent can be manipulated by changing a concentration and/or type of the biologically active agent in the emulsion. In still further aspects, the release of the biologically active agent can be manipulated by changing size of an initial emulsion droplet that is introduced into a subject. In some aspects, the formation of the solid matrix from the liquid delivery system can occur over period of several hours. In yet other aspects, the formation of the solid matrix is instantaneous. In certain aspects, the compositions and methods described herein can be carefully controlled and manipulated to prevent an initial burst of the biologically active agent to further prevent toxic side effects.

Accordingly, the droplet is present in an emulsion.

Accordingly, disclosed herein is a kit comprising: a) a first mixture comprising a pharmaceutically acceptable polar liquid and a fully or nearly fully dissolved biodegradable polymer; b) a second mixture comprising a pharmaceutically acceptable non-polar liquid; and c) a mixing device configured to mix the first mixture and second mixture, thereby forming a droplet comprising the biodegradable polymer.

Further disclosed herein is a kit comprising: a) a biologically active agent; b) a pharmaceutically acceptable carrier capable of delivering the biologically active agent; c) a first mixture comprising a pharmaceutically acceptable polar liquid and a fully or nearly fully dissolved biodegradable polymer; d) a second mixture comprising a pharmaceutically acceptable non-polar liquid; e) and one or more mixing device configured to mix the biologically active agent and the pharmaceutically acceptable carrier with the first mixture to form a primary suspension and then to mix the primary suspension with the second mixture, thereby forming at least one droplet comprising the biologically active agent.

It is understood that the a) a biologically active agent and b) a pharmaceutically acceptable carrier capable of delivering the biologically active agent can be present in the kit as a mixture or separate parts. Accordingly, in one aspect, the a) a biologically active agent and b) a pharmaceutically acceptable carrier capable of delivering the biologically active agent can be present in the kit as a mixture. In another aspect, the a) a biologically active agent and b) a pharmaceutically acceptable carrier capable of delivering the biologically active agent can be present in the kit as separate parts.

In some aspects, the mixing of the first and the second mixture can be performed in any mixing device known in the art. In certain aspects, the mixing of the first and second mixture in the mixing device results in forming a droplet comprising the biodegradable polymer.

In certain aspects, the size of the droplet can depend on the mixing device. In certain aspects, the size of the droplet can be in the range from about 10 nm to about 500 μm, including exemplary values of about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 50 μm, about 100 μm, about 150 μm, about 200 μm, about 250 μm, about 300 μm, about 350 μm, about 400 μm, and about 450 nm. In other aspects, the droplet size can be any size in the range between any two of the above states values. For example, the droplet size can be from about 100 nm to about 1000 nm, or from about 500 nm to about 100 μm, or from about 1 μm to about 500 μm.

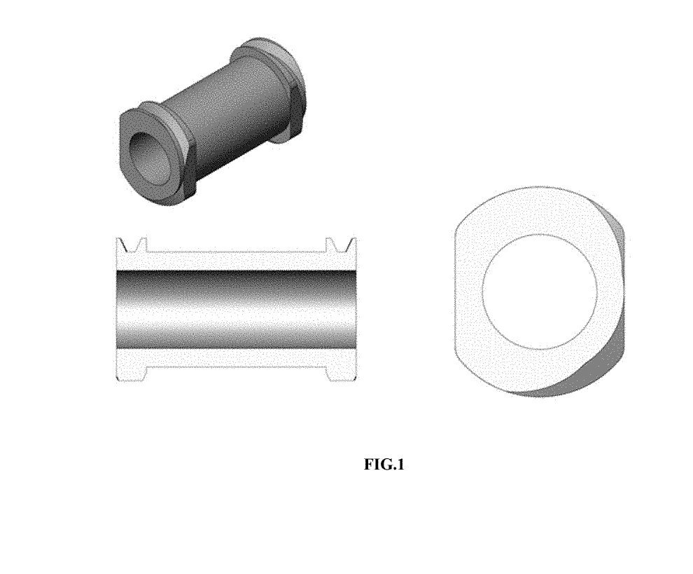

In some aspects, the mixing device present in the kit can comprise a luer lock connector, a static mixer, or an orifice plate, or a combination thereof. The luer lock connectors are known in the art. The exemplary unmodified luer lock connector is shown in FIG. 1. It is understood that the luer lock connector can have any opening size that can be chosen by one of ordinary skill in the art to achieve a predetermined droplet size in the final emulsion. It is further understood that the size of the mixing device can be utilized to manipulate the droplet size or shape and as a result to manipulate the drug release. For example and without limitation, it is understood that a smaller opening will result in a smaller droplet.

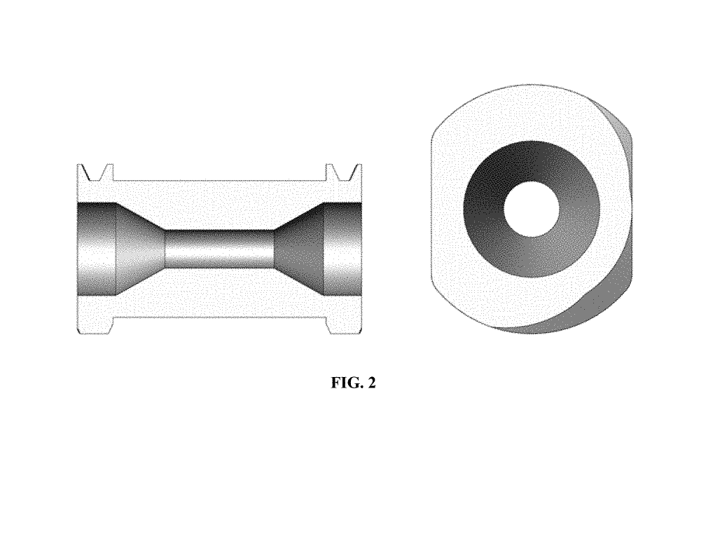

In some aspects, the inner diameter (I.D.) of the mixing device can stay constant or vary throughout the mixing device (FIG. 2) In some aspects, the ratio between the smallest inner diameter and the largest inner diameter can be from about 1:100 to about 1:2, including exemplary values of about 1:90, about 1:80, about 1:70, about 1:60, about 1:50, about 1:40, about 1:30, about 1:20, about 1:10, about 1:9, about 1:8, about 1:7, about 1:6, about 1:5, about 1:4, and about 1:3. In some aspects, the ratio between the smallest inner and the largest inner diameter can have any value between two foregoing values. In yet other aspects, it is understood that the ratio between the smallest inner and largest inner diameter can be regulated based on a desirable size of the droplet or a disirable shape of the droplet.

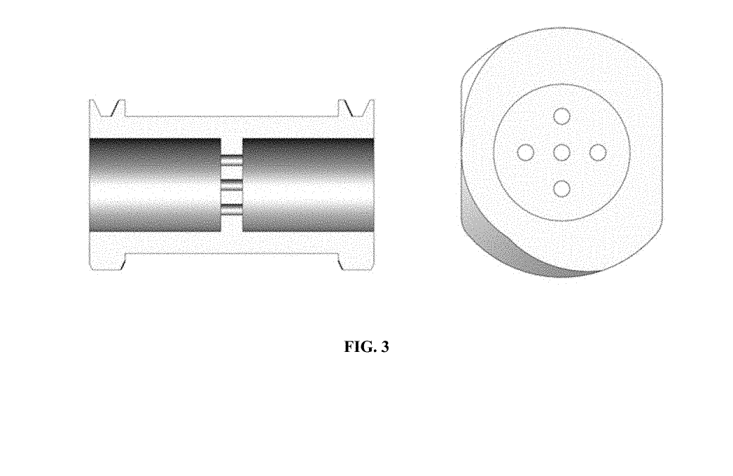

In some aspects, the mixer is plate-type mixer, such as an orifice plate mixer. In the plate type design mixing is accomplished through intense turbulence in the flow. In these aspects, a perforated orifice plate can be placed within luer connector (FIG. 3). The orifice plates are known in the art. It is understood that the orifice plate can comprise any number of openings (holes) to ensure an adequate mixing and achieving a predetermined droplet size in the final emulsion. It is further understood that the size of the openings in the mixing device can be utilized to manipulate the droplet size or dropleet shape and as a result to manipulate the drug release.

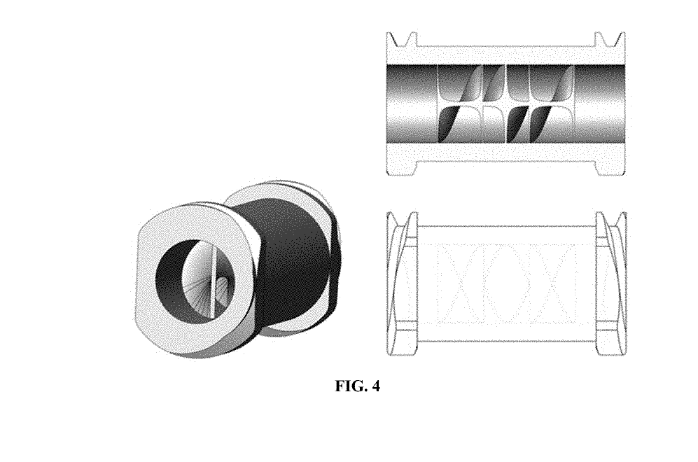

In certain aspects, the mixing device comprises a static mixer. The static mixers are also known in the art. In some aspects, the static mixer are located within the luer. The exemplary static mixer placed into the luer connector is shown in FIG. 4. As one of ordinary skill in the art would readily appreciate, the static mixers are precision engineered motionless mixing devices that allow for the continuous blending of fluids. With no moving parts, static mixers utilize the energy of flow stream to generate consistent and reliable mixing. In some aspects, the static mixer can comprise a device comprising of mixer elements contained in a cylindrical tube or squared housing. In certain aspects, the housed-elements design of the static mixer can comprise a series of baffles. It is understood that the static mixer can have any size allowing to achieve a predetermined droplet size or shape in the final emulsion. The length of the static mixer can be chosen depending on the degree of mixing required to achieve a final emulsion with the predetermined droplet sizes. It is further understood that the size of the mixing device can be utilized to manipulate the droplet size and as a result to manipulate the drug release.

In yet other aspects, the kit further comprises an injection device configured to inject the droplet comprising the biodegradable polymer into a subject, thereby forming a microparticle in vivo in the subject. In some aspects, the injection device is configured to be positioned on the mixing device. In yet other aspects, the injection device is a mixing device. In yet other aspects, the injection device is a separate from the mixing device. In yet other aspects, the injection device can comprise a needle. Needle can be utilized to introduce the mixture into the patient's body.

In some aspects, the mixing and injection of the first mixture and second mixture produces a microparticle comprising the biodegradable polymer with an average diameter of less than 250 μm, including exemplary values of less than about 225 μm, less than about 200 μm, less than about 175 μm, less than about 150 μm, less than about 125 μm, less than about 100 μm, less than about 75 μm, less than about 50 μm, less than about 25 μm, less than about 10 μm, less than about 5 μm, or less than about 1 μm. In other aspects, the microparticle can be any size in the range between any two of the above states values. For example, the microparticle can be from about 1 μm to about 50 μm, or from about 25 μm to about 100 μm, or from about 50 μm to about 250 μm.

It is further understood that the microparticle size can be easily controlled by the mixing device and can be a determining factor in release of the biologically active agent into the patients' body. It is further understood that different target areas within the subject may require different size of the microparticles. In some aspects, the current invention allows one of ordinary skill in the art to manipulate the size of the microparticles depending on the target areas. In yet other aspects, the current invention allows one of ordinary skill in the art carefully control the drug release from the microparticle. It is understood to those of ordinary skill in the art that one of the key factors effecting release of a biologically active agent from a microparticle is the size of the particle. This is explained by the increased surface area to volume ratio of small particles compared to the reduced surface area to volume ratio of large particles.

In certain aspects, the pharmaceutically acceptable polar liquid can comprise any aqueous or nonaqueous polar liquids that can be safely used for pharmaceutical purposes. In some aspects, the pharmaceutically acceptable polar liquid is water. In other aspects, the pharmaceutically acceptable polar liquid is a pharmaceutically acceptable polar organic solvent. It is understood that the pharmaceutically acceptable polar liquid is capable of fully dissolving or nearly fully dissolving a biodegradble polymer.

It is understood that in the aspects, wherein the pharmaceutically acceptable polar liquid comprises a polar organic solvent, upon contact with an aqueous medium, such as water or body fluids, the polar organic solvent can diffuse or leach into the surrounding aqueous medium leading to precipitation of a biodegradable polymer that is substantially insoluble in aqueous fluids to form a solid microparticle.

In some aspects, suitable polar organic solvents used in the first mixture are those which are biocompatible, pharmaceutically-acceptable, and will fully or nearly fully dissolve the biodegradable polymeric material. According to the invention, the pharmaceutically acceptable polar solvent can have solubility in aqueous medium, ranging from miscible to dispersible and is capable of diffusing into an aqueous medium, for example, tissue fluids, such as blood serum, lymph, cerebral spinal fluid (CSF), and saliva. In certain aspects, the pharmaceutically acceptable organic polar solvent can also be biocompatible with surrounding environment.

In some aspects, the organic polar solvent can have water solubility ranging from a high water solubility i.e., from those forming about 20% by weight solution in water to those completely miscible in all properties, including exemplary values of about 30% by weight, about 40% by weight, about 50% by weight, about 60% by weight, about 70% by weight, about 80% by weight, about 90% by weight, and about 100% by weight solution in water. In other aspects, the organic polar solvent can form a solution in water having concentration in the range between any two of the above stated values. For example, the organic polar solvent described herein can form a solution in water having concentration of about 30% by weight to about 60% by weight, or about 40% by weight to about 90% by weight.

In other aspects, the organic polar solvent can have a low water solubility i.e., those forming solution with less than about 20% by weight of the solution in water, including exemplary values of about 1% by weight, about 3% by weight, about 5% by weight, about 8% by weight, about 10% by weight, about 12% by weight, about 15% by weight, and about 18% by weight. In other aspects, the organic polar solvent can form a solution in water having concentration in the range between of any two of the above stated values. For example, the organic polar solvent described herein can form a solution in water having concentration of about 1% by weight to about 10% by weight, or about 5% by weight to about 18% by weight.

It is further understood and without being bound by any theory, a polar organic solvent with a “high” water solubility can rapidly diffuse or dissipate from the delivery composition into the surrounding aqueous fluids. In contrast, a polar organic solvent having a “low” solubility in an aqueous medium will dissipate slowly in the aqueous medium.

In certain aspects, the pharmaceutically acceptable polar organic solvent comprise substituted heterocyclic compounds such as N-methyl-2-pyrrolidone (NMP) and 2-pyrrolidone; C₂ to C₁₀ alkanoic acids such as acetic acid, lactic acid and heptanoic acid; esters of hydroxy acids such as methyl lactate, ethyl lactate, alkyl citrate and the like; monoesters of polycarboxylic acids such as monomethyl succinate acid, monomethyl citric acid and the like, ether alcohols such as 2-ethoxyethanol, ethylene glycol dimethyl ether, glycofurol and glycerol formal; alcohols such as ethanol and propanol; polyhydroxy alcohols such as propylene glycol, polyethylene glycol (PEG), glycerin (glycerol), 1,3-butyleneglycol, and isopropylidene glycol (2,2-dimethyl-1,3-dioxolone-4-methanol; solketal; dialkylamides such as dimethylformamide, dimethylacetamide; dimethylsulfoxide (DMSO) and dimethylsulfone; lactones such as ε-caprolactone and butyrolactone; cyclic alkyl amides such as caprolactam; aromatic amides such as N,N-dimethyl-m-toluamide, and 1-dodecylazacycloheptan-2-one; and mixtures and combinations thereof. For example and without limitation, the solvents can include N-methyl-2-pyrrolidone, 2-pyrrolidone, dimethylsulfoxide, ethyl lactate, glycofurol, glycerol formal, and isopropylidene glycol. In yet other aspects, the polar organic solvent comprises of dimethyl sulfoxide (DMSO), or N-Methyl-2-pyrrolidone (NMP), or a combination thereof

In yet other aspects, the pharmaceutically acceptable polar organic solvents disclosed herein comprise low water solubility solvents. Examples of low water soluble solvents include without limitation, esters of carbonic acid and alkyl alcohols such as propylene carbonate, ethylene carbonate and dimethyl carbonate alkyl esters of mono-, di-, and tricarboxylic acids, such as 2-ethyoxyethyl acetate, ethyl acetate, methyl acetate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as methyl ethyl ketone, tetrahydrofuran as well as other carbonyl, ether, carboxylic ester, amide and hydroxy containing liquid organic compounds having some solubility in water. In some aspects, due to the known biocompatitibility and pharmaceutical acceptance, propylene carbonate, ethyl acetate, triethyl citrate, isopropyl myristate, and glyceryl triacetate can be utilized.

In certain aspects, the mixtures of the foregoing high and low water solubility solvents can be used in the first mixture.

In some aspects, useful polymers can comprise biodegradable polymers. In yet other aspects the biodegradable polymer can comprise polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. In certain aspects, the biodegradable polymer can be a polysaccharide, including modified or substituted forms of polysaccharides. Examples include without limitation maltodextrin, including both modified and substituted forms of a maltodextrin, starches, glycogen, cellulose, chitin, chitosan, dextrin, dextrans, glycans, glucans, hyalurorans, and modified or substituted versions thereof.

In yet other aspects, the polymer can comprise poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers.

In a further aspect, other durable polymers can include without limitation ethylene-vinyl acetate co-polymer, polytetrafluoroethylene, polypropylene, polyethylene, and the like. In yet other aspects, other suitable polymers can include without limitation silicones and polyurethanes.

In yet further aspects, the biodegradable polymer can comprise a polyester, polyethylene glycol, polyamide, polyalkyl-α-cyano acrylate, polyorthoester, polylactic acid, polyurethane, or polyacrylamides, or a combination thereof.

In a still further aspect, the polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

In a still further aspect, useful biocompatible polymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biocompatible polymers are those that comprise one or more residues of lactide, glycolide, caprolactone, or a combination thereof.

In one aspect, useful biodegradable polymers are those that comprise one or more blocks of hydrophilic or water soluble polymers, including, but not limited to, polyethylene glycol, (PEG), or polyvinyl pyrrolidone (PVP), in combination with one or more blocks another biocompabible or biodegradable polymer that comprises lactide, glycolide, caprolactone, or a combination thereof.

In yet other aspects, the biodegradable polymer comprises polylactic acid (PLA), polycaprolactone (PCL), polyglycolide (PGA), polylactide-co-glycolide (PLGA), or polyethylene glycol (PEG), or a combination hereof.

In specific aspects, the biodegradable polymer can comprise one or more lactide residues. To that end, the polymer can comprise any lactide residue, including all racemic and stereospecific forms of lactide, including, but not limited to, L-lactide, D-lactide, and D, L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic D, L-lactide, L-lactide, and D-lactide polymers are commercially available. The L-polymers are more crystalline and resorb slower than D, L-polymers. In addition to copolymers comprising glycolide and D, L-lactide or L-lactide, copolymers of L-lactide and D, L-lactide are commercially available. Homopolymers of lactide or glycolide are also commercially available.

In the aspects, when the biodegradable polymer is poly(lactide-co-glycolide), poly(lactide), or poly(glycolide), the amount of lactide and glycolide in the polymer can vary. In a further aspect, the biodegradable polymer can comprise from 0 to 100 mole %, about 40 to 100 mole %, about 50 to 100 mole %, about 60 to 100 mole %, about 70 to 100 mole %, or about 80 to 100 mole % lactide and from 0 to 100 mole %, 0 to about 60 mole %, about 10 to about 40 mole %, about 20 to about 40 mole %, or about 30 to about 40 mole % glycolide, wherein the amount of lactide and glycolide is 100 mole %. In a further aspect, the biodegradable polymer can be poly(lactide), about 95:5 poly(lactide-co-glycolide), about 85:15 poly(lactide-co-glycolide), about 75:25 poly(lactide-co-glycolide), about 65:35 poly(lactide-co-glycolide), or about 50:50 poly(lactide-co-glycolide), where the ratios are mole ratios.

In a further aspect, the polymer can be a poly(caprolactone) or a poly(lactide-co-caprolactone). In one aspect, the polymer can be a poly(lactide-caprolactone), which, in various aspects, can be about 95:5 poly(lactide-co-caprolactone), about 85:15 poly(lactide-co-caprolactone), about 75:25 poly(lactide-co-caprolactone), about 65:35 poly(lactide-co-caprolactone), about 50:50 poly(lactide-co-caprolactone), about 40:60 poly(lactide-co-caprolactone), about 25:75 poly(lactide-co-caprolactone), about 10:90 poly(lactide-co-caprolactone), or about 5:95 poly(lactide-co-caprolactone), where the ratios are mole ratios.

It is understood that any combination of the aforementioned biodegradable polymers can be used, including, but not limited to, copolymers thereof, mixtures thereof, or blends thereof. Likewise, it is understood that when a residue of a biodegradable polymer is disclosed, any suitable polymer, copolymer, mixture, or blend, that comprises the disclosed residue, is also considered disclosed. When multiple residues are individually disclosed (i.e., not in combination with another), it is understood that any combination of the individual residues can be used.

In some aspects, the biodegradable polymer can be present in the first mixture in any desired weight %. For example, the polymer can be present from about 1% to about 90% by weight, including without limitation, about 5%, 10%, 15%, 20%, 30%, 40%, 50%, 60%, 70%, or 80% by weight. In other aspects, the biodegradable polymer can be present in the first mixture in the range between at any two of the above stated values. For example, the biodegradable polymer can be present in about 10% by weight to about 60% by weight, or about 30% by weight to about 90% by weight.

In some aspects, the biodegradable polymer can have a molecular weight from about 100 Daltons to about 2,000,000 Daltons, including exemplary values of about 200 Daltons, about 500 Daltons, about 1,000 Daltons, about 2,000 Daltons, about 3,000 Daltons, about 4,000 Daltons, about 5,000 Daltons, about 6,000 Daltons, about 7,000 Daltons, about 8,000 Daltons, about 9,000 Daltons, about 10,000 Daltons, about 20,000 Daltons, about 30,000 Daltons, about 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, and about 1,900,000 Daltons. In yet other aspects, the biodegradable polymer can have a molecular weight in the range between any two of the above stated values. For example, the biodegradable polymer can have a molecular weight between about 100 Dalton to about 100,000 Dalton, or about 500 Dalton to about 500,000 Dalton, or from about 500,000 Dalton to about 1,500,000 Dalton.

In certain aspects, a second mixture comprises a pharmaceutically acceptable non-polar liquid. It is understood that the pharmaceutically acceptable non-polar liquid can comprise any non-polar liquid that can be used in pharmaceutical compositions or patients' treatments without adversely affecting the patients' health. In certain aspects, the pharmaceutically acceptable non-polar liquids comprise oils, low melting waxes, fats, lipids, and any other pharmaceutically acceptable substance that is lipophilic, substantially insoluble in water body, fluid, and is biodegradable and/or eliminatable by natural processes of a patient's body. In some aspects, the pharmaceutically acceptable non-polar liquids comprise oils of plants such as vegetables and seeds. Examples can include without limitation oils made from corn, sesame, cannoli, soybean, castor, peanut, olive, arachis, maize, almond, flax, safflower, sunflower, rape, coconut, palm, babassu, and cottonseed oil; waxes such as carnoba wax, beeswax, and tallow; fats such as triglycerides, lipids such as fatty acids and esters, and liposomes such as red cell ghosts and phospholipid layers.

In yet other aspects, the pharmaceutically acceptable oil is a plant oil comprising corn oil, sesame oil, cannoli oil, soybean oil, castor oil, peanut oil, olive oil, arachis oil, maize oil, almond oil, flax oil, safflower oil, sunflower oil, rape oil, coconut oil, palm oil, babassu oil, or cottonseed oil, or a combination thereof. In yet other aspects, the plant oil comprises soybean oil.

In some aspects, the first mixture can further comprise a biologically active agent. In certain aspects, the biologically active agent present in the first mixture is combined with the second mixture by mixing to form a biologically active droplet. As used herein, a biologically active agent is an agent that is capable of providing a local or systemic biological, physiological or therapeutic effect in the body of a patient.

In some aspects, the biologically active agent can be present in an amount effective to provide the desired level of biological, physiological, pharmacological and/or therapeutic effect in the patient. It is understood that there is generally no critical upper limit on the amount of the biologically active agent that can be included in the composition. For optimal performance, the concentration of the bioactive agent should not be so high that the controlled release composition cannot effectively control the rate of release of the bioactive agent. The lower limit of the amount of bioactive agent incorporated into the first mixture depends on the activity of the bioactive material and the period of time desired for treatment. Generally, one skilled in the art of formulations can determine the appropriate amount of biologically active agent to incorporate into the first mixture as a function of the patient's condition, the physical characteristics of the biologically active agent and the prescribed treatment regimen for the malcondition of the patient.

In yet other aspects, the biologically active agent can be present in the first mixture from about 1% to about 90% by weight, including without limitation, about 5%, 10%, 15%, 20%, 30%, 40%, 50%, 60%, 70%, or 80% by weight. In yet other aspects, the biologically active agent can be present in the range between any two of the above stated values. For example, the biologically active agent can be present from about 5% to about 40%, or about 30% to about 70% by weight.

In some aspects, a wide variety of biologically active agents can be used. In one aspect, the biologically active agents can be biologically active agents, i.e., biologically active agents that can be released from the droplet or the microparticle into adjacent tissues or fluids of a subject. In some aspects, the biologically active agents are at least very slightly water soluble. In yet other aspects, the biologically active agents are moderately water soluble. The biologically active agents can include salts of the active ingredient. In some aspects, the biologically active agents can be acidic, basic, or amphoteric salts. In yet other aspects, the biologically active agents can be nonionic molecules, polar molecules, or molecular complexes capable of hydrogen bonding. In still further aspects, the biologically active agents can be included in the compositions in the form of, for example, an uncharged molecule, a molecular complex, a salt, an ether, an ester, an amide, polymer drug conjugate, or other form to provide the effective biological or physiological activity.

Examples of biologically active agents that incorporated into systems herein include, but are not limited to, peptides, proteins such as hormones, enzymes, antibodies, antibody fragments and the like, nucleic acids such as aptamers, iRNA, DNA, RNA, antisense nucleic acid or the like, antisense nucleic acid analogs or the like, low-molecular weight compounds, or high-molecular-weight compounds. In some aspects, the biologically active agents contemplated for use include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-vascular growth agents, anti-vascular endothelial growth agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, vascular growth agents, vascular endothelial growth agents, uterine relaxants, vitamins, or antigenic materials.

In yet other aspects, the biologically active agents can include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

In certain aspects, the representative drugs that can be used as biologically active agents can include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

It is further understood that the first mixture can comprise a large number of biologically active agents either singly or in combination. Other bioactive agents can include but are not limited to analgesics such as acetaminophen, acetylsalicylic acid, and the like; anesthetics such as lidocaine, xylocalne, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, antibody fragments, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

In certain aspects, the biologically active agents can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthraces, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainjluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In yet other aspects, the biologically active agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the bioactive agent can be a combination of Rifampicin (Rifampin in U.S.) and Minocycline.

In certain aspects, the biologically active agent can be present as a component in a pharmaceutical composition. Pharmaceutical compositions can be conveniently prepared in a desired dosage form, including, for example, a unit dosage form or controlled release dosage form, and prepared by any of the methods well known in the art of pharmacy. In general, pharmaceutical compositions are prepared by uniformly and intimately bringing the biologically active agent into association with a liquid carrier or a finely divided solid carrier, or both. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other pharmaceutically acceptable carriers or components that can be mixed with the bioactive agent can include, for example, a fatty acid, a sugar, a salt, a water-soluble polymer such as polyethylene glycol, a protein, polysachamide, or carboxmethyl cellulose, a surfactant, a plasticizer, a high- or low-molecular-weight porosigen such as polymer or a salt or sugar, or a hydrophobic low-molecular-weight compound such as cholesterol or a wax.

In some aspects, examples of suitable biologically active agents include substances capable of preventing an infection systemically or locally at the defect site, for example, anti-inflammatory agents such as hydrocortisone, and prednisone; antibacterial agents such as penicillin, cephalosporins, bacitracin, tetracycline, doxycycline, gentamycin, quinolines, neomycin, clindamycin, kanamycin, azithromycin and metronidazole; antiparasitic agent such as quinacrine, chloroquine, and vidarbine; antifungal agents such as nystatin; antiviral agent such as acyclovir, ribarivin, and interferons; analgesic agents such as salicylic acid, acetaminophen, ibuprofen, naproxen, piroxicam, flurbiprofen, and morphine; local anesthetics such as cocaine, lidocaine, bupivacaine and benzocaine; immunogens (vaccines) for simulating antibodies against hepatitis, influenza, measles, rubella, tetanus, polio, and rabies; peptides such as leuprolide acetate (an LH-RH agonist), nafarelin, and ganirelix.

In other aspects, substances, or metabolic precursors thereof, which are capable of promoting growth and survival of cells and tissues or augmenting the functioning of cells can also be used, for example and without limitation, a nerve growth promoting substance, such as a ganglioside or a nerve growth factor; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-α), transforming growth factor-β, (TGF-β), epidermal growth factor (EGF), fibroblast growth factor (FGF), and interleukin-1 (IL-1); an osteoinductive agent or bone growth promoting substance such a bone chips or demineralized bone material; and antineoplastic agents such as methotrexate, 5-fluorouracil, adriamycin, vinblastine, cisplatin, paclitaxel, floxuridine, tumor-specific antibodies conjugated to toxins, and tumor necrosis factor.

In still further aspects, the biological active agent can comprise genes which encode biologically useful proteins, such as growth hormone, growth hormone releasing factor, pituitary factors, adrenal factors, pancreatic factors, interferon factors, prostaglandin releasing factors and the like. In yet other aspects, the biological active agent can comprise cells, for example, cells such as fibroblasts, osteoblasts, chondrocytes, retinal pigmented epithelia, epithelia, β-islet cells, mesenchymal stem cells, and other cells within the body.

In yet other aspects, the biologically active agent comprises anti-inflammatory agent, an antibacterial agent, an antifungal agent, an analgesic agent, an anesthetic agent, an immunogenic, a vaccine, an antineoplastic agent, a growth or survival gene, a hormone, a cardiovascular agent, an anti-ulcer agent, a bronchial agent, a central nervous system agent, a peptide, a gene, a gene fragment, or an insertion vector carrying agent or gene fragments, or a combination thereof.

In some aspects, to deliver the biologically active agent into the body of the patients, the first and the second mixtures are mixed in the mixing device configured to mix the first mixture and second mixture, thereby forming a droplet. In yet other aspects, the kit disclosed herein further comprises an injection device configured to inject the droplet comprising the biodegradable polymer into a subject, thereby forming a microparticle in vivo in the subject. It is understood that in the aspects, wherein the first mixture comprises a pharmaceutically acceptable polar liquid and the second mixture comprises a pharmaceutically acceptable non-polar liquid an emulsion or dispersion can be formed upon mixing the first and the second mixtures. It is further understood that the emulsion involves a stable mixture of two or more immiscible liquids which form a suspension or dispersion having a continuous phase and a dispersed phase.

It is further understood that in some aspects, the emulsion can further comprise emulsion stabilizers. Non-limiting examples of suitable stabilizers include surfactants or emulsification aids such as poly(vinyl alcohol), PVA, or polysorbate surfactants or poloxamers. The emulsion can further comprise an emulsifier, which aids in the formation of the emulsion or double-emulsion. A non-limiting example of an emulsifier is the surfactant Tween 80 or the poloxamer Pluronics F168. In yet further aspects, the emulsions can further comprise other additives such as a polar organic solvent, a cosolvent, a buffer, a salt, a sugar, or a combination thereof. In some aspects, the emulsion stabilizers are present in the second mixture.

### 3. METHODS

Disclosed herein is a method of producing a microparticle in vivo in a subject comprising the steps of: a) mixing a first mixture comprising a pharmaceutically acceptable polar liquid and a fully or nearly fully dissolved biodegradable polymer in the a pharmaceutically acceptable polar liquid and a second mixture comprising a pharmaceutically acceptable non-polar liquid in a mixing device, thereby forming a droplet comprising the biodegradable polymer; and b) prior to forming the microparticle, injecting the droplet into a subject with an injection device, thereby forming the microparticle in vivo in the subject.

Also disclosed herein is a method of producing a microparticle in vivo in a subject comprising the steps of: a) mixing a biologically active agent with a first solution comprising a pharmaceutically acceptable carrier to form a first composition; b) mixing the first composition with a second solution in a first mixing device, wherein the second solution comprises a biodegradable polymer and a pharmaceutically acceptable polar liquid to form a primary emulsion; c) mixing the primary emulsion with a second mixture comprising a pharmaceutically acceptable non-polar liquid in a second mixing device to form a droplet within a secondary emulsion; d) prior to forming the microparticle, injecting the droplet within the secondary emulsion into a subject with an injection device, thereby forming the microparticle in vivo in the subject.

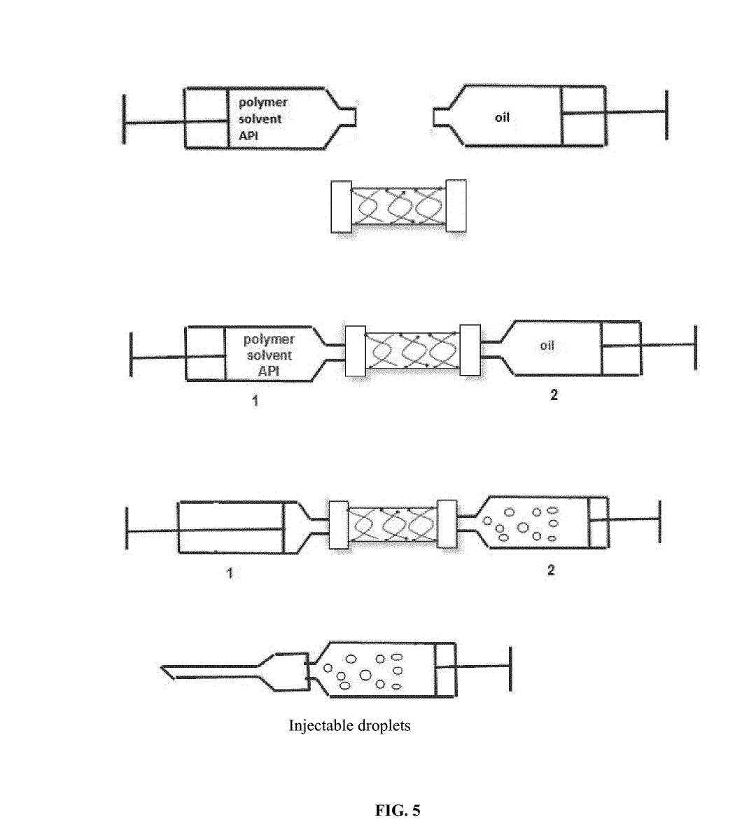

In some aspects, the first mixture can comprise the foregoing biologically active agents, biodegradable polymers and polar and non-polar solvents. In other aspects, the biologically active agent is incorporated into the first mixture simultaneously with or immediately prior to mixing with the second mixture (FIG. 5). In these aspects, a first container, for example and without limitation, a first syringe (1) comprising a first mixture comprising a biodegradable polymer, a solvent capable of fully or nearly fully dissolving the biodegradable polymer, and a biologically active agent (or alternatively active pharmaceutical ingredient (API)) is mixed with a second mixture comprising a pharmaceutically acceptable non-polar liquid such as any of mentioned above oils, for example. The second mixture is delivered in a second container, for example a second syringe (2). The two mixtures are mixed utilizing any of the foregoing mixers. In some aspects, the mixing can be achieved by pushing the first mixture present in the first syringe thereby the mixing device to the second syringe to form an emulsion. In some other aspects, the mixing can be further continued by returning the formed emulsion into the mixing device and then to the first syringe to ensure an efficient mixing. The mixing steps can be repeated a number of times, wherein the specific number of repetitions be easily determined by the one of ordinary skill in the art depending on desirable outcomes. In certain aspects, the emulsion prepared by the mixing and comprising droplets comprising a biodegradable polymer and a biologically active agent can be injected into a subject, thereby forming the microparticle in vivo in the subject.

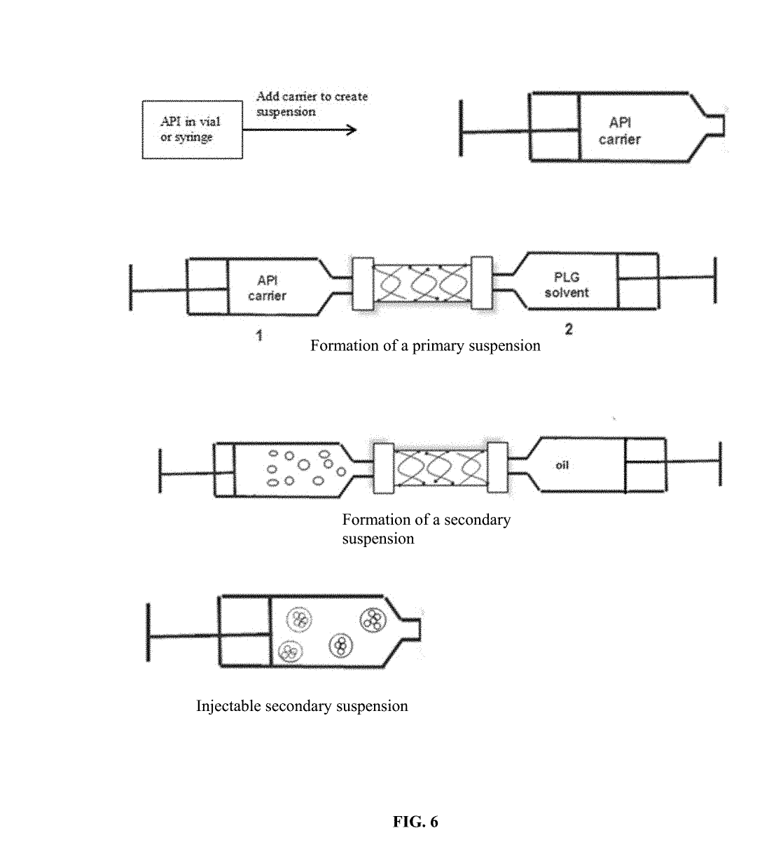

In some aspects, the biologically active agent can be incorporated into the first mixture by any techniques known in the art. In some aspects, the biologically active agent is incorporated into the first mixture prior to mixing with the second mixture (FIG. 6). In some exemplary aspects, the biologically active agent or alternatively an active pharmaceutical ingredient (API) can be provided in a vial or syringe. The API can be further dissolved or dispersed in a carrier solvent. In some aspects, the carrier solvent can be any previously described a pharmaceutically acceptable polar or non-polar liquid. In yet other aspects, the carrier solvent is a pharmaceutically acceptable polar liquid. The composition comprising the API and the carrier are further mixed with a composition comprising a biodegradable polymer and a pharmaceutically acceptable solvent capable fully or nearly fully to dissolve the biodegradable polymer. The mixing can be performed in a first mixing device that can comprise any foregoing mixing devices to form a primary suspension comprising an API, a carrier, a biodegradable polymer and a solvent. In some aspects, a carrier and a solvent can be same or different. The formed primary suspension is further mixed utilizing a second mixing device with the second mixture comprising a pharmaceutically acceptable non-polar liquid, for example any foregoing non-polar liquids or oils to form a secondary emulsion that can be further injected into the subject to form microparticles in situ. It is understood that the first and second mixing devices can be the same or different. It is also understood that the mixing can be repeated a number of times, and a number of mixing repetitions can be easily determined by one of ordinary skills in the art.

In some aspects, to prepare the droplet comprising the biodegradable polymer, any known technique for suspending, dispersing or emulsifying one material within another can be used. Additionally, the biologically active material may be sprayed, aerosolized, or otherwise converted or comminuted into small droplets or particles which are then combined with the controlled release formulation. In some aspects, the biologically active agent can be dissolved or dispersed into the polymer solution prior to forming the emulsion.

In yet one aspect, the droplet is formed by use the mixing device present in the disclosed kit. In some aspects, the steps a) and b) of the disclosed method can be performed simultaneously (or in quick or nearly simultaneous) succession (for example, by using a multi-chamber syringe). In such an aspect, the droplets are injected almost immediately upon formation. For example, within 2 min, 1 min, 30 sec, or 15 sec of formation. In other aspects, the steps a) and b) of the disclosed method can be performed subsequently.

In some aspects, a first appropriately sized syringe containing a first flowable mixture is connected to a second appropriately sized syringe filled with a second flowable mixture. In certain aspects, the first and the second syringes are connected by a luer lock device serving as a mixing device. In yet other aspects, the first and the second syringes are connected by a static mixer serving as a mixing device. In still further aspects, the first and the second syringes are connected with a luer lock device comprising a static mixer, an orifice plate, or a combination thereof. In further aspects, the plunger of the first syringe containing the first mixture is actuated to transport an appropriate amount of the first mixture into the mixing device. In yet still further aspects, the plunger of the second syringe containing the second mixture is actuated to transport an appropriate amount of the second mixture in the mixing device. In certain aspects, the mixing device comprises, a luer lock, a static mixer, or an orifice plate, or a combination thereof. In yet other aspects, the mixing device can comprise a luer lock comprising a static mixer, or an orifice plate, or a combination thereof. In certain aspects, the plungers of the two syringes are reciprocated a number of times to agitate the mixture within the mixing device and convert the mixture into small suspended droplets. In yet other aspects, the first and the second mixtures are mixed by any known in the art methods. In still further aspects, the previously formed mixture is connected to a mixing device described herein, such as luer lock, for example, and is further agitated to form suspended droplets. In yet other aspects, the injection device is connected to the mixing device. In these aspects, the droplet is injected into a subjected with the injection device, thereby forming the microparticle in vivo in the subject.

It is understood that in certain aspects, the administration of can be accomplished by any convenient technique. For example, the formulation can be applied by brushing, spraying, extruding, dripping, injecting, or painting. Yet in other aspects, the administration is achieved by injection device comprising a needle.

In some aspects, the subject to receive the formed droplets can be any subject requiring therapeutic or scientific treatments. In some aspects, the subject is human. In yet another aspect, the subject is an animal. In yet other aspects, the subject is any research tissue or a system existing outside of the human or animal body.

### Aspects

In view of the disclosure herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A kit comprising: a) a first mixture comprising a pharmaceutically acceptable polar liquid and a fully or nearly fully dissolved biodegradable polymer; b) a second mixture comprising a pharmaceutically acceptable non-polar liquid; and c) a mixing device configured to mix the first mixture and second mixture, thereby forming at least one droplet comprising the biodegradable polymer.

Aspect 2: The kit of aspect 1, wherein the kit further comprises an injection device configured to inject the droplet comprising the biodegradable polymer into a subject, thereby forming a microparticle in vivo in the subject.

Aspect 3: The kit of aspects 1 or 2, wherein the first mixture further comprises a biologically active agent.

Aspect 4: The kit of any one of aspects 1-3, wherein the kit further comprises d) a biologically active agent and e) a pharmaceutically acceptable carrier.

Aspect 5: The kit of aspect 3, wherein the biologically active agent comprises an antiinflammatory agent, an antibacterial agent, an antifungal agent, an analgesic agent, an anesthetic agent, an immunogen, a vaccine, an antineoplastic agent, a growth or survival agent, a hormone, a cardiovascular agent, an anti-ulcer agent, a bronchial agent, a central nervous system agent, a peptide, a gene, a gene fragment, or an insertion vector carrying a gene or gene fragment, or a combination thereof.

Aspect 6: The kit of any one of aspects 1-5, wherein the pharmaceutically acceptable polar liquid is a pharmaceutically acceptable polar organic solvent.

Aspect 7: The kit of aspect 6, wherein the polar organic solvent comprises dimethyl sulfoxide (DMSO), or N-Methyl-2-pyrrolidone (NMP), or a combination thereof.

Aspect 8: The kit of any one of aspects 1-7, wherein the biodegradable polymer comprises a polyester, polyethylene glycol, polyamide, polyalkyl-α-cyano acrylate, polyorthoester, polyurethane, polyacrylamides, polylactic acid (PLA), polycaprolactone (PCL), polyglycolide (PGA), polylactide-co-glycolide (PLGA), or polyethylene glycol (PEG), or a combination thereof.

Aspect 9: The kit of any one of aspects 1-8, wherein the pharmaceutically acceptable non-polar liquid is a pharmaceutically acceptable oil.

Aspect 10: The kit of aspect 9, wherein the pharmaceutically acceptable oil is a plant oil comprising of corn oil, sesame oil, cannoli oil, soybean oil, castor oil, peanut oil, olive oil, arachis oil, maize oil, almond oil, flax oil, safflower oil, sunflower oil, rape oil, coconut oil, palm oil, babassu oil, or cottonseed oil, or a combination thereof.

Aspect 11: The kit of aspect [00117], wherein the plant oil comprises soybean oil.

Aspect 12: The kit of any one of aspects 1-11, wherein the mixing and injection of the first mixture and second mixture produces a microparticle comprising the biodegradable polymer with an average diameter of less than about 250 μm.

Aspect 13: The kit of any one of aspects 1-12, wherein the mixing device comprises a luer lock connection, a static mixer, an orifice, or a combination thereof.

Aspect 14: The kit of any one of aspects 2-13, wherein the injection device comprises a needle.

Aspect 15: A method of producing a microparticle in vivo in a subject comprising the steps of: a) mixing a first mixture comprising a pharmaceutically acceptable polar liquid and a fully or nearly fully dissolved biodegradable polymer in the a pharmaceutically acceptable polar liquid and a second mixture comprising a pharmaceutically acceptable non-polar liquid in a mixing device, thereby forming a droplet comprising the biodegradable polymer; and b) prior to forming the microparticle, injecting the droplet into a subject with an injection device, thereby forming the microparticle in vivo in the subject.

Aspect 16: The method of aspect 15, wherein steps a) and b) are performed nearly simultaneously.

Aspect 17: The method of aspects 15 or 16, wherein the microparticles form an implant in vivo in the subject.

Aspect 18: The method of any one of aspects 15-17, wherein the injection is in a joint of the subject.

Aspect 19: The method of any one of aspects 15-18, wherein the first mixture further comprises a biologically active agent.

Aspect 20: The method of aspect 19, wherein the biologically active agent comprises an antiinflammatory agent, an antibacterial agent, an antifungal agent, an analgesic agent, an anesthetic agent, an immunogen, a vaccine, an antineoplastic agent, a growth or survival agent, a hormone, a cardiovascular agent, an anti-ulcer agent, a bronchial agent, a central nervous system agent, a peptide, a gene, a gene fragment, and an insertion vector carrying a gene or gene fragment, or a combination thereof.

Aspect 21: The method of aspects 12, wherein the pharmaceutically acceptable polar liquid is a pharmaceutically acceptable polar organic solvent.

Aspect 22: The method of aspect 16, wherein the polar organic solvent comprises of DMSO, or NMP, or a combination thereof.

Aspect 23: The method of any one of aspects 15-22, wherein the biodegradable polymer comprises a polyester, polyethylene glycol, polyamide, polyalkyl-α-cyano acrylate, polyorthoester, polylactic acid, polyurethane, or polyacrylamides, or a combination thereof.

Aspect 24: The method of any one of aspects 15-23, wherein the biodegradable polymer comprises polylactic acid (PLA), polycaprolactone (PCL), polyglycolide (PGA), polylactide-co-glycolide (PLGA), or polyethylene glycol (PEG), or a combination thereof.

Aspect 25: The method of any one of aspects 15-24, wherein the pharmaceutically acceptable non-polar liquid is a pharmaceutically acceptable oil.

Aspect 26: The method of aspect 18, wherein the pharmaceutically acceptable oil is a plant oil comprising corn oil, sesame oil, cannoli oil, soybean oil, castor oil, peanut oil, olive oil, arachis oil, maize oil, almond oil, flax oil, safflower oil, sunflower oil, rape oil, coconut oil, palm oil, babassu oil, or cottonseed oil, or a combination thereof.

Aspect 27: The method of aspect [00133], wherein the plant oil comprises soybean oil.

Aspect 28: The method of any one of aspects 15-27, wherein the method produces a microparticle comprising the biodegradable polymer with an average diameter of less than 250 μm.

Aspect 29: The method of any one of aspects 15-28, wherein the mixing device comprises a luer lock connector, a static mixer, or an orifice plate, or a combination thereof.

Aspect 30: The method of any one of aspects 15-29, wherein the mixing injection comprises a needle.

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. A kit comprising:
a) a first mixture comprising a pharmaceutically acceptable polar liquid and a fully or nearly fully dissolved biodegradable polymer;
b) a second mixture comprising a pharmaceutically acceptable non-polar liquid; and
c) a mixing device configured to mix the first mixture and second mixture, thereby forming at least one droplet comprising the biodegradable polymer.

2. The kit of claim 1, wherein the kit further comprises an injection device configured to inject the droplet comprising the biodegradable polymer into a subject, thereby forming a microparticle in vivo in the subject.

3. The kit of claim 1, wherein the first mixture further comprises a biologically active agent.

4. The kit of claim 1, wherein the kit further comprises d) a biologically active agent and e) a pharmaceutically acceptable carrier.

5. The kit of claim 3, wherein the biologically active agent comprises an antiinflammatory agent, an antibacterial agent, an antifungal agent, an analgesic agent, an anesthetic agent, an immunogen, a vaccine, an antineoplastic agent, a growth or survival agent, a hormone, a cardiovascular agent, an anti-ulcer agent, a bronchial agent, a central nervous system agent, a peptide, a gene, a gene fragment, or an insertion vector carrying a gene or gene fragment, or a combination thereof.

6. The kit of claim 1, wherein the pharmaceutically acceptable polar liquid is a pharmaceutically acceptable polar organic solvent.

7. The kit of claim 6, wherein the polar organic solvent comprises dimethyl sulfoxide (DMSO), or N-Methyl-2-pyrrolidone (NMP), or a combination thereof.

8. The kit of claim 1, wherein the biodegradable polymer comprises a polyester, polyethylene glycol, polyamide, polyalkyl-α-cyano acrylate, polyorthoester, polyurethane, polyacrylamides, polylactic acid (PLA), polycaprolactone (PCL), polyglycolide (PGA), polylactide-co-glycolide (PLGA), or polyethylene glycol (PEG), or a combination thereof.

9. The kit of claim 1, wherein the pharmaceutically acceptable non-polar liquid is a pharmaceutically acceptable oil.

10. The kit of claim 1, wherein the mixing device comprises a luer lock connection, a static mixer, an orifice, or a combination thereof.

11. The kit of claim 2, wherein the injection device comprises a needle.

12. A method of producing a microparticle in vivo in a subject comprising the steps of:
a) mixing a first mixture comprising a pharmaceutically acceptable polar liquid and a fully or nearly fully dissolved biodegradable polymer in the a pharmaceutically acceptable polar liquid and a second mixture comprising a pharmaceutically acceptable non-polar liquid in a mixing device, thereby forming a droplet comprising the biodegradable polymer; and
b) prior to forming the microparticle, injecting the droplet into a subject with an injection device, thereby forming the microparticle in vivo in the subject.

13. The method of claim 12, wherein steps a) and b) are performed simultaneously.

14. The method of claim 12, wherein the first mixture further comprises a biologically active agent.

15. The method of claim 14, wherein the biologically active agent comprises an antiinflammatory agent, an antibacterial agent, an antifungal agent, an analgesic agent, an anesthetic agent, an immunogen, a vaccine, an antineoplastic agent, a growth or survival agent, a hormone, a cardiovascular agent, an anti-ulcer agent, a bronchial agent, a central nervous system agent, a peptide, a gene, a gene fragment, and an insertion vector carrying a gene or gene fragment, or a combination thereof.

16. The method of claim 12, wherein the pharmaceutically acceptable polar liquid is a pharmaceutically acceptable polar organic solvent.

17. The method of claim 16, wherein the polar organic solvent comprises of DMSO, or NMP, or a combination thereof.

18. The method of claim 12, wherein the biodegradable polymer comprises a polyester, polyethylene glycol, polyamide, polyalkyl-α-cyano acrylate, polyorthoester, polyurethane, polyacrylamides, polylactic acid (PLA), polycaprolactone (PCL), polyglycolide (PGA), polylactide-co-glycolide (PLGA), or polyethylene glycol (PEG), or a combination thereof.

19. The method of claim 12, wherein the pharmaceutically acceptable non-polar liquid is a pharmaceutically acceptable oil.

20. A method of producing a microparticle in vivo in a subject comprising the steps of:
a) mixing a biologically active agent with a first solution comprising a pharmaceutically acceptable carrier to form a first composition;
b) mixing the first composition with a second solution in a first mixing device, wherein the second solution comprises a biodegradable polymer and a pharmaceutically acceptable polar liquid to form a primary emulsion;
c) mixing the primary emulsion with a second mixture comprising a pharmaceutically acceptable non-polar liquid in a second mixing device to form a droplet within a secondary emulsion; and
d) prior to forming the microparticle, injecting the droplet within the secondary emulsion into a subject with an injection device, thereby forming the microparticle in vivo in the subject.
